(19) United States
(12) Patent Application Publication
(10) Pub. No.: US 2020/0190135 A1
(43) Pub. Date: Jun. 18, 2020

(54) LASSO STRUCTURES AND THEIR SYNTHESIS

(71) Applicant: ChromaCon AG, Zurich (CH)

(72) Inventors: Jeffrey BODE, Zürich (CH); Fumito SAITO, Zürich (CH)

(73) Assignee: ChromaCon AG, Zurich (CH)

(21) Appl. No.: 16/349,064

(22) PCT Filed: Nov. 9, 2017
(86) PCT No.: PCT/EP2017/078689

§ 371 Date: May 10, 2019

(30) Foreign Application Priority Data
Nov. 16, 2016 (EP) 16199170.8

(51) Int. Cl. C07K 1/00; C07K 7/06

(52) U.S. Cl. CPC C07K 1/00; C07K 7/06

(57) ABSTRACT

A method for the synthesis of a molecular lasso structure in which a linear moiety is covalently attached to a cyclic moiety and with its free end is partially threaded through the orifice formed by the cyclic moiety, including the following steps: 1) provision of a cyclic and a first linear structural element and establishing conditions in which the first linear structural element is threaded through the orifice of the cyclic moiety; 2) covalently attaching a stopper element to one terminal end of the linear structural element; 3) separating unthreaded from threaded molecular assemblies by chemical or physical separation; and 4) reacting the threaded molecular assemblies with a second linear structural element so that it is covalently attached to the first linear structural element at its end opposite to the end where the stopper is attached, and so that the second linear structural element is covalently attached to the cyclic moiety.

### TECHNICAL FIELD

The present invention relates to the synthesis of new lasso structures and lasso structures obtained using the method.

### PRIOR ART

Natural lasso peptides can have remarkable thermal and proteolytic stability compared to linear peptides due to their particular threaded structure. Attempts to stabilize linear peptides by grafting into lasso peptide structures so far had limited success. The loop of lasso peptides has been grafted with small peptide sequences; however the in vivo processing machinery is limiting the size, position, and sequences of peptides that can be grafted. To overcome the shortcomings of a biological system, the chemical synthesis of lasso peptides has been attempted, albeit unsuccessfully, not displaying the correct lasso structure.

Countless linear peptides have been found, which have remarkable medicinal potential, but have the important and inherent drawbacks of often being unstructured and unstable in vivo. Several approaches towards solving the inherent problems of peptides while maintaining their medicinal activity have been reported, including cyclization, backbone N-methylation, backbone “stapling”, or incorporation of unnatural amino acids.

Lasso compounds consist of a macrocycle with a covalently attached linear structure, which is threaded through the macrocycle. There are some naturally occurring lasso peptides, which appear in such a conformation, for example Microcin J25 (MccJ25). Certain lasso peptides, including MccJ25, have been found to have remarkable thermal and proteolytic stability compared to linear and branched peptides. Thus, it would be desirable to produce self-chosen peptide sequences in lasso shape in order to improve structural, thermal, and proteolytic stability. Several general approaches towards this goal have been explored, but all hitherto existing strategies have important drawbacks.

The generation of hybrid lasso peptides with heterologous sequences grafted into the loop structure of natural lasso peptide by means of genetically engineering the changes has been shown to work for small peptides. For example, the integrin-targeting RGD-sequence has been incorporated into MccJ25. Indeed, the proteolytic stability was increased by this measure. However, there are very narrow limitations to this approach, among others due to the natural synthesis of lasso peptides. Lasso peptides are ribosomally synthesized in linear form and post-translationally modified by enzymes to bring them into their distinct lasso form. Thus, the extent of variability is limited, among others by the substrate scope of the involved enzymes. It has been found that the size of loop can only be varied to a small extent, for example 1-2 amino acid residues. Moreover, certain residues within the natural sequence cannot be modified at all. Furthermore, modifications often decrease expression yields. Mature synthetic methods and production strains for lasso peptides do not exist. Notably, it is not yet fully understood which natural lasso peptides possess increased stability and which do not. Not even all of the to date known 38 natural lasso peptides possess thermal stability. Thus, not all natural templates are useful, and some modifications, even if technically feasible, may produce lasso compounds of lower stability. Recently it was shown that not only the plugging residues of the linear threaded peptide part are important for thermal stability of lasso peptides, but that also the residues in the macrocyclic moiety play a role.

Another possible approach consists of purifying biologically synthesized lasso peptides and subsequently modifying them by chemical means. It is conceivable that the loop sequence of such a designed molecule could be opened and grafted with a peptide by chemical ligation or other synthetic means in vitro. To date there are no reports of this approach. The disadvantages of this theoretical approach greatly overlap with what is described above. The lack of achieving substantial expression levels of lasso peptides due to the complexity and inherent limitations of the lasso-forming processing machinery in vivo is limiting an in-vivo production approach at scale. Furthermore, the scope of post-expression modifications is limiting as well.

Yet another approach comprises a completely chemical de novo synthesis of lasso peptides. For other template types, such as cyclic peptides, this approach has been reported. Compared to the aforementioned approaches, there are no inherent limitations regarding the scalability, or the sequence composition and length. However, lasso peptides in the relevant threaded conformation are to date synthetically inaccessible. Therefore, it is not possible to simply build lasso peptides with desired sequences and correct structure by chemical synthesis.

A related approach is based on constructing hybrid molecules between non-peptidic chemical scaffolds and therapeutic peptide or peptidic sequences. For this approach there are no inherent limitations regarding the scalability, or the sequence composition and length, albeit, decreasing yields are expected with increasing sequence length. There is one prior report for the synthesis of a lasso compound consisting of a non-peptidic template and the incorporated tripeptide glycine-glycine-glycine. However, this particular reported approach has several important limitations. The synthetic strategy consists of first covalently conjugating a macrocyclic molecule with a peptide containing thread, yielding a covalent branched cyclic compound with a peptide. In a second step a stopper moiety is attached to the distal end of the linear peptide containing part. The stopper moiety is needed to prevent de-threading by fully drawing the linear moiety through the macrocycle. The lasso formation is achieved in the next step via so-called “self-entanglement by bond rotation” (see FIG. 1). This means that the loop of the lasso is formed by rotating the bond between the macrocycle and the linear branch in a way that the linear thread is carried through the macrocycle.

This approach, however, is only possible, if the full peptide sequence incorporated in the threading part is slim enough to fit through the macrocycle. Thus, amino acids with bulky side chains cannot be incorporated. In fact, only amino acids without any side chain have been demonstrated to be suitable for that approach. Neither can other molecular building blocks with spacious groups be incorporated. Additionally, the self-entanglement by bond rotation is reversible. To prevent de-threading of the lasso compound by reverse self-entanglement by bond rotation, a second internal stopper needs to be attached. During the lasso formation and before fixation by attaching the additional internal stopper, only nonpolar solvents, low temperatures and low concentrations may be used, because otherwise no or only a small fraction of the compound will be in lasso configuration. For example in methanol at room temperature, 100% of the compound is unthreaded. To avoid unthreading, an additional internal stopper molecule has to be attached. The potential maximum yield corresponds to the fraction of threaded lasso compound under the reaction conditions. The reported yield even with this very simple tri-glycine peptide is consequently very low (4% alone for the final locking step). The properties of the purified peptide containing lasso compound have only been studied in methanol. There are no data on potential benefits of the lasso structure on the peptide properties, including heat or protease stability. Thus this approach has severe limitations on scalability and applicability.

Clavel et al (Molecules, 18 (9), 11553-11575 disclose the synthesis of a peptide—containing lasso molecular switch by a self entanglement strategy involving rotaxane interlocking.

Hiroshi et al (Chemistry, A European Journal, 22(19) 6624-6630) discloses rational design for rotaxane synthesis through intermolecular slippage and control of activation energy by rigid axial length.

WO2012146729 provides novel templates for peptide grafting. The template is a modified lasso peptide wherein an amino acid sequence of the wildtype lasso peptide comprising one to five amino acids is substituted by another amino acid sequence comprising one to five amino acids. In the modified lasso peptides, any site of the wildtype lasso peptide can be substituted by another amino acid sequence comprising one to five amino acids. The term “another amino acid sequence” refers to any amino acid sequence that differs from the amino acid sequence of the respective site in the wildtype lasso peptide. The modified lasso peptides according to the present invention are produced by applying the natural maturation machinery for the processing of lasso peptide precursor variants. These variants are generated on the DNA level of the precursor genes by site-directed mutagenesis. The lasso peptide precursor proteins are heterologously coexpressed with the two processing enzymes and the ex-port/immunity protein which transform the precursor into the modified lasso variant that is secreted into the culture supernatant from which the modified lasso peptide is extracted and purified. The modified lasso peptides according to the present invention can be used for peptide grafting.

WO2014036213 provides are astexin-1, astexin-2 and astexin-3 lasso peptides, which are based on sequences identified in Asticaccaulis excentricus, and methods of making and using same. Astexin-1 is highly polar, in contrast to many lasso peptides that are primarily hydrophobic, and has modest antimicrobial activity against Caulobacter crescentus, a bacterium related to Asticaccaulis excentricus. The solution structure of astexin-1 was determined, revealing a unique topology that is stabilized by hydrogen bonding between segments of the peptide. Astexins-2 and -3 are intracellular lasso peptides.

### SUMMARY OF THE INVENTION

Therefore, it is desirable to be able to synthetically incorporate linear molecules, such as peptide sequences, including such with therapeutic or targeting prospects, into synthetic lasso forming templates. Herein, we present an approach making use of synthetic cyclic scaffolds, e.g. rotaxane scaffolds, which allow for the incorporation of linear molecules, including therapeutic and targeting peptides, and thereby convey them structural stability and protect them from thermal and/or proteolytic degradation. The present invention relates to a method for the generation of lasso compounds comprising a synthetic scaffold for incorporation of peptide or peptido-mimetic motifs. The resulting hybrid lasso structures can additionally be used to convey a chemical and/or biological payload to a target in vivo.

The aim of the present invention is to provide a tool to graft a linear molecule into an overall lasso-type structure. Thereby, the flexibility of the formerly linear molecule is constrained, which, among others, for medicinal applications, will lead to increased binding affinities due to reduced entropic binding penalties, or for peptides will lead to increased stability against degradation by, for example, proteases. Naturally occurring lasso peptides represent an effective strategy to stabilize α-peptides, which would otherwise be quickly degraded by proteases. Research groups have developed a method to replace one to five amino acid residues in the natural lasso peptide Microcin J25 (MccJ25). The resulting hybrid lasso peptide stabilized the otherwise unstable heterologous RGD peptide sequence, increased the binding affinity to integrins, and enhanced its biological effects. The grafting has been achieved by intracellular biosynthesis. A purely chemical approach for implementing arbitrary peptides into lasso structures is, to date, not available. The present invention solves this shortcoming and demonstrates the chemical synthesis of several lasso compounds incorporating synthetic peptides. As such, the approach is scalable and can be used to obtain lasso compounds on any desired scale. This is achieved by synthetically incorporating target molecules into template molecules, thereby forming a lasso structure. A lasso structure consists of a cyclic structure with a covalently attached linear structure, which is threaded through the previously mentioned cyclic structure. The region of the linear structure between the attachment point to the cyclic structure and the threaded part is the Loop Region. Liquid chromatography and nuclear resonance spectroscopy (NMR) can be employed to prove the lasso structure. Thermal, enzymatic, and in-serum degradation studies confirm the anticipated increase in peptide stability.

Further folding into a more complex structure, for example through side chain cross-linking of the grafted molecule yielding a compact three-dimensional rigid structure, is also possible.

Additionally to the advantages mentioned above, these structures serve to stabilize peptide sequences in the lasso scaffold and using them for targeting in vivo, and/or using such a lasso scaffold to transport a chemical or biological payload to a target in vivo. Combinations of targeting and transport of payloads are known well with antibody-drug conjugates and smaller protein domains have been used with the same effect. As peptides are not stable in vivo and structural flexibility often diminishes their binding affinities, they have not reached the importance of bigger proteinogenic moieties such as antibodies. On the other hand, small targeting molecules will have a much better tissue penetration than the much larger antibodies and protein domains used as target reagents. The aim of the present invention thus is to provide a mean to produce lasso compounds with an easily modifiable Loop Region and the opportunity to further functionalize the lasso compound with orthogonal chemistry to carry a chemical or biological payload.

Both macrocyclic and loop region of the lasso structure can be varied (see below). The loop region is accessible for interaction with other molecules, for example proteins. Thus, it is ideally suited to accommodate molecular structures, which act like drugs. The approach in the present invention allows the incorporation of virtually any peptide sequence. This was demonstrated by incorporating peptide sequences Cys-Nle-Trp-Phe-Arg-His-Tyr-Lys-Leu, Cys-Gly-Leu-Arg-Arg-Leu-Phe-Ala-Asn-Leu, Cys-Lys-Asn-Phe-Phe-Trp-Lys-Thr-Phe-Thr-Ser-Leu into lasso compounds.

The circumference of the cyclic structure is limited on the lower end by the prerequisite that the smaller end of a linear molecular structure can be threaded through its interior. The linear structure needs to have a thinner end, and a wider end terminated by a “stopper” moiety. The upper limit for the circumference of the cyclic structure is given by the prerequisite that the stopper may not be able to thread through the interior of the cyclic structure in significant amounts.

The circumference of the cyclic structure may thus not be strictly limited by the number or type of atoms making up the cyclic structure. Also the geometry and flexibility of the geometry may influence the upper and lower limits of the size of the cyclic structure. Furthermore, size of macrocycle and stopper moiety need to match, to prevent significant amounts of de-threading.

Further, different macrocycles can be employed. As stated above, the size of the stopper moiety has to be large enough to prevent de-threading in significant amounts. In preferred embodiments of the present invention, the cyclic structure is represented by 1,4,7,10,13,16,19-Benzoheptaoxacycloheneicosin-21-ethyleneamine, alternatively by 1,4,7,10,13,16,19-Heptaoxacyclodocosan-21-amine, and alternatively by 1,4,7,10,13,16,19-Heptaoxa-22-azacyclotetracosane.

Following the present invention, also stoppers, which could be further conjugated with molecular entities such as drug payloads, could be incorporated. In such embodiments the lasso moiety could positively influence physicochemical and/or pharmacokinetic properties, including, but not limited to, absorption, distribution, metabolism, excretion, toxicity (ADME-Tox), for the molecular entity conjugated to the lasso via the stopper moiety.

The general synthetic strategy is outlined in FIG. 2 B. A linear molecule with two orthogonal attachment points is added to a macrocyclic molecule with a branched attachment point. The conditions (solvent, temperature, pH) are chosen to favor a threaded conformation. In a second step, a stopper moiety is attached to one end of the linear molecule. The unthreaded part can then easily be removed and be discriminated from the properly threaded structure by mass spectrometry. The loop region, which may contain a peptide, is then attached to modifiable branching point and then finally also to the threaded linear molecule. The resulting molecule forms a lasso.

The resulting lasso compounds may have improved thermal and proteolytic activity. The preferred embodiments show good thermal stability as well as superior stability against various proteases and in human serum. Also applications outside of the medicinal field can profit from the advantageous properties gained by implementing such lasso structures.

Effect of macrocycle structure and size: The macrocycles of natural lasso peptide have 7-9 amino acids. Smaller rings are undesirable, because this would prevent threading. On the upper end the ring sizes are limited by the size of the internal and terminal stoppers. Too big rings, or respectively too small stoppers (see below), can lead to “de-threading”.

Calculated from typical bond lengths therefore preferably the following can be calculated as suitable ring sizes: natural peptides: 7-9 residues, meaning 30-39 Å; 22-atom crown ether ring: 32 Å; 24 atom crown ether ring: 35 Å; 21-atom catechol crown ether ring: 30 Å. It may further be advantageous to provide an internal stopper moiety to prevent de-threading via reverse self-entanglement by bond rotation. However, as usually peptide residues with side chains will be used, these side chains already can fulfil this requirement. More generally speaking, the present invention relates to a method for the synthesis of a molecular lasso structure consisting of a cyclic moiety and of a linear moiety, wherein the linear moiety is covalently attached to the cyclic moiety and with its free end is partially threaded through the orifice formed by the cyclic moiety. The proposed method is including the following steps, preferably in given order:

1) provision of a cyclic moiety and of a first linear structural element and establishing conditions in which at least a fraction of the first linear structural element is threaded through the orifice of the cyclic moiety;

2) covalently attaching a stopper element preventing de-threading of the first linear structural element to one terminal end of the first linear structural element;

3) separating unthreaded moieties from threaded moieties, in particular separating cyclic moieties and first linear structural elements from threaded molecular assemblies, in which threaded molecular assemblies consist of the first linear structural element with said stopper element threaded through the cyclic moiety, by chemical and/or physical separation and using essentially only the threaded molecular assemblies for further reaction;

4) reacting the threaded molecular assemblies with a bifunctional second linear structural element so that the second linear structural element is covalently attached to the first linear structural element at or close to its end opposite to the end where the stopper is attached, and so that the second linear structural element is directly or indirectly covalently attached to the cyclic moiety.

As mentioned above, preferably steps 1)-4) are carried out in the given order. However also different reaction pathway is possible leading to the same final product. In this different pathway, also object of the present invention, a bifunctional second linear structural element it is reacted with a cyclic moiety so that the second linear structural element is directly or indirectly covalently attached to the cyclic moiety. This attachment of the bifunctional 2nd linear structural element with the cyclic moiety takes place, prior to, during, or after any of steps 1) or 2). Then step 3) is carried out. In this process, in step 4) the threaded molecular assemblies are reacted with the bifunctional second linear structural element directly or indirectly covalently attached to the cyclic moiety so that the second linear structural element is further directly or indirectly covalently attached to the first linear structural element at or close to its end opposite to the end where the stopper is attached.

In any case, the bifunctional 2nd linear structural element can either be directly covalently attached to the cyclic moiety, or it can be indirectly attached, i.e. covalently attached to a moiety covalently attached to the cyclic moiety.

The above-mentioned step 3) of separation can also be omitted in cases where essentially only threaded molecular assemblies result from step 1). Or step 3) of separating unthreaded moieties from threaded assemblies can be carried out at the end of the process, when the molecular lasso structure has been generated by going through steps 1), 2), 4), preferably in given order. In other words the method can be carried out by going through steps 1), 2), 4), preferably in given order, including the possibility of first attaching the 2nd linear structural element directly or indirectly covalently to the cyclic moiety, and to then carry out a separation step 3) separating unthreaded lasso peptides from threaded lasso peptides.

According to a preferred embodiment, in step 4) the bifunctional second linear structural element is attached with one free end to the corresponding free end of the first linear structural element, and the bifunctional second linear structural element is attached with the other free end to a functional group provided on the cyclic moiety, wherein preferably the functional group provided on the cyclic moiety is provided as a side chain, particularly preferably an amino-group carrying side chain.

The cyclic moiety is preferably selected to be a ring comprising polyether building blocks, the building blocks preferably being selected from the group consisting of at least 1 of: 1,3 or 1,2 propylene-diol, 1,2 ethylene-diol, methylene-diol, benzene-1,2-diol. These building blocks can be unsubstituted or substituted, and preferably, if substituted, the building blocks are substituted by amino, methylamino, ethylamino groups so as to allow for covalent attachment of the second linear structural element in the final step 4). Preferably such side chains are protected in steps 1-3 and only unprotected for the linking reaction to the second linear structural element in the final step 4.

According to yet another preferred embodiment, the cyclic moiety is based exclusively on building blocks selected from the group consisting of at least 1 of: 1,3 or 1,2 propylene-diol, 1,2 ethylene-diol, methylene-diol, benzene-1,2-diol. Alternatively the cyclic moiety can be based on these building blocks as well as further based on one, preferably only one building block selected from the group consisting of: methanolamine, ethanolamine, propanolamine, ethylenediamine, 1,3-diaminopropane, 2-aminophenol, benzene-1,2-diamine.

The cyclic moiety can be selected from the group consisting of 1,4,7,10,13,16,19-Benzoheptaoxacycloheneicosin-21-ethyleneamine, alternatively by 1,4,7,10,13,16,19-Heptaoxacyclodocosan-21-amine, and 1,4,7,10,13,16,19-Heptaoxa-22-azacyclotetracosane or mixtures thereof.

Generally speaking, preferably the cyclic moiety is a crown ether, including catechol crown ether.

The cyclic moiety according to yet another preferred embodiment has a diameter of its orifice in the range of 25-60 Å or 20-45 Å, preferably in the range of 30-40 Å.

The first linear structural element in a portion to be located opposite to the stopper element preferably comprises a bulky structural element, preferably in the form of a side chain, preferably including an aromatic group, wherein further preferably the bulky structural element is selected from the group consisting of phenylalanine, tryptophan, tyrosine, (3-amino acids containing bulky side chains, bulky branched-alkyl chains wherein the bulky sidechains and/or the bulky branched-alkyl chains are preferably selected from the group consisting of leucine, valine, and isoleucine. In case a 21 membered ring (e.g. compound 2) is used, a branched-alkyl chain can be a large enough stopper. Thus, leucine, valine, and isoleucine are also suitable options.

The first linear structural element may comprise at least one heteroatom in a central region thereof allowing for the establishment of conditions in step 1) under which a non-covalent bond is established in the orifice between the cyclic moiety and said heteroatom, wherein preferably the non-covalent bond is selected from the group consisting of hydrogen bond, ionic bond, or a combination thereof and wherein further preferably the heteroatom under the said conditions carries a partial or full positive charge for interaction with heteroatoms in the cyclic moiety, said heteroatoms in the cyclic moiety preferably being selected from the group consisting of oxygen atoms, sulfur atoms, nitrogen atoms.

The stopper element can be selected to be a structural moiety having an aromatic group, preferably based on benzoic acid, unsubstituted or substituted with one, two, or more substituents, such as aliphatic groups, including methyl or ethyl groups, and/or halogen atoms, and/or ether groups, including polyether groups such as PEG-substituents on phenols, ether, ester, nitrile, and nitro groups. The stopper element is preferably selected from the group consisting of: mono fluoro benzoic acid, benzoic acid, methylbenzoic acid, dimethylbenzoic acid, bromo benzoic acid, iodo benzoic acid, and 2-phenylacetic acid, which are unsubstituted or substituted, in the latter case preferably with groups including ether, ester, nitrile, and nitro groups.

For reaction in step 2) the stopper element can be provided as an activated compound, including systems with alkyne, maleimide, alkene, azide, and aldehyde/ketone structural elements, or as an acyltrifluoroborate, when used with suitable reaction partners.

The first linear structural element according to yet another preferred embodiment comprises at least one amino acid building block and/or at least one building block selected from the group consisting of methanolamine, ethanolamine, propanolamine, ethylenediamine, 1,3-diaminopropane, 2-aminophenol, benzene-1,2-diamine.

The first structural element may further comprise an activated end for reaction with the stopper element, and a second non-activated end, wherein preferably the activated end takes the form of a group selected from the following group nitrile, azido, thiol, alkene, in particular C1-C6 alkene, alkyne in particular C1-C6 alkyne, hydroxylamine.

The second linear structural element is preferably a linear peptide, preferably having 3-15 amino acids, more preferably having 4-10 or 5-8 amino acids, wherein preferably the second linear structural element is a linear peptide with known therapeutic effect.

The present invention furthermore relates to a molecular lasso structure consisting of a cyclic moiety and of a linear moiety, wherein the linear moiety is covalently attached to the cyclic moiety and is partially threaded through the orifice formed by the cyclic moiety, obtained using a method as described above.

Last but not least the present invention relates to a molecular lasso structure according to claim 11 as a medicament.

Further embodiments of the invention are laid down in the dependent claims.

### BRIEF DESCRIPTION OF THE DRAWINGS

Preferred embodiments of the invention are described in the following with reference to the drawings, which are for the purpose of illustrating the present preferred embodiments of the invention and not for the purpose of limiting the same. In the drawings,

FIG. 1 shows self-entanglement by bond rotation;

FIG. 2 shows synthetic strategies towards lasso compounds with incorporated peptides;

FIG. 3 shows a proof of concept synthesis of a lasso compound with incorporated peptide. Abbreviations: Fmoc: 9-fluorenylmethoxycarbonyl; DMSO: dimethyl sulfoxide; RT: room temperature; MPAA: 4-mercaptophenylacetic acid; TCEP: Tris(2-carboxyethyl)phosphine;

FIG. 4 shows a branched-cyclic and non-threaded analogue of L1, B1;

FIG. 5 shows the peptide substrate scope of the approach;

FIG. 6 shows trypsin digestion of lasso compounds leading to rotaxanes, proving the successful threading of the initial compounds;

FIG. 7 shows alternative macrocyclic moieties employable in lasso compound synthesis;

FIG. 8 shows stopper moieties 1 and 15 for prevention of de-threading of lasso compounds; stopper moiety 1 is suitable for use with macrocycle 2, while stopper moiety 15 is additionally suitable to prevent de-threading of lasso compounds based on the macrocycle moieties 13 and 14;

FIG. 9 shows structures of lasso compounds L2 and L3 comprising peptide sequences, stopper moiety 15, and macrocycle 13 or 14, respectively;

FIG. 10 shows branched cyclic analogues B2 and B3;

FIG. 11 shows a cyclic peptide derivative C1;

FIG. 12 shows a stability test versus chymotrypsin degradation;

FIG. 13 shows a stability test versus trypsin degradation;

FIG. 14 shows a stability test versus proteinase K degradation;

FIG. 15 shows a N- and C-terminally unmasked linear peptide R1; and

FIG. 16 shows a stability test in human serum.

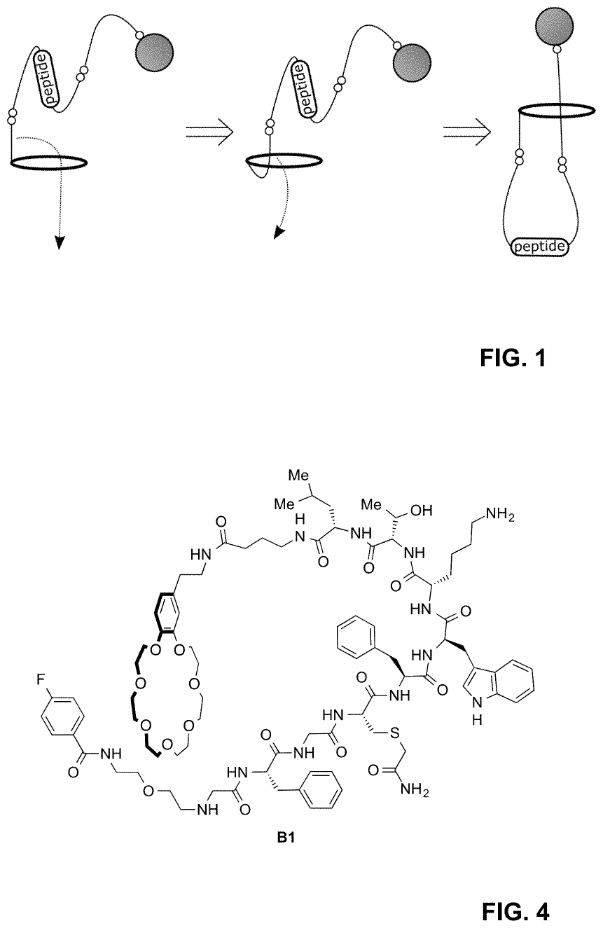

### DESCRIPTION OF PREFERRED EMBODIMENTS

### Example 1

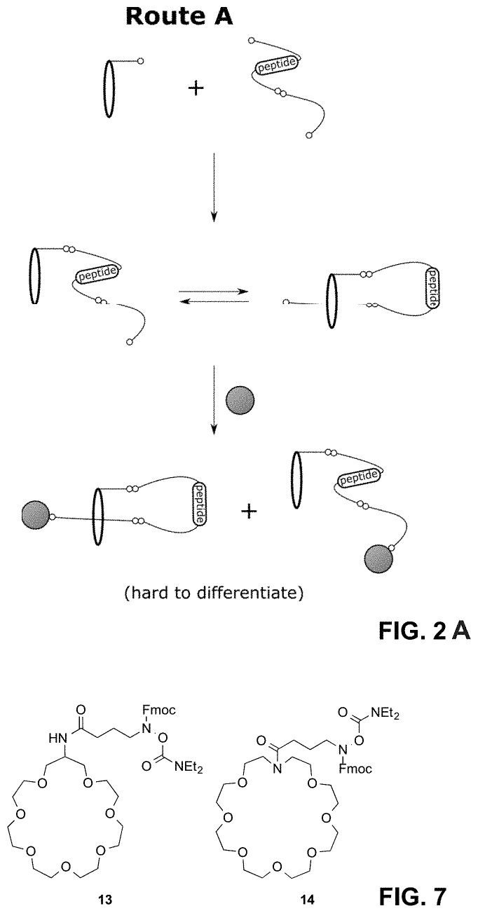

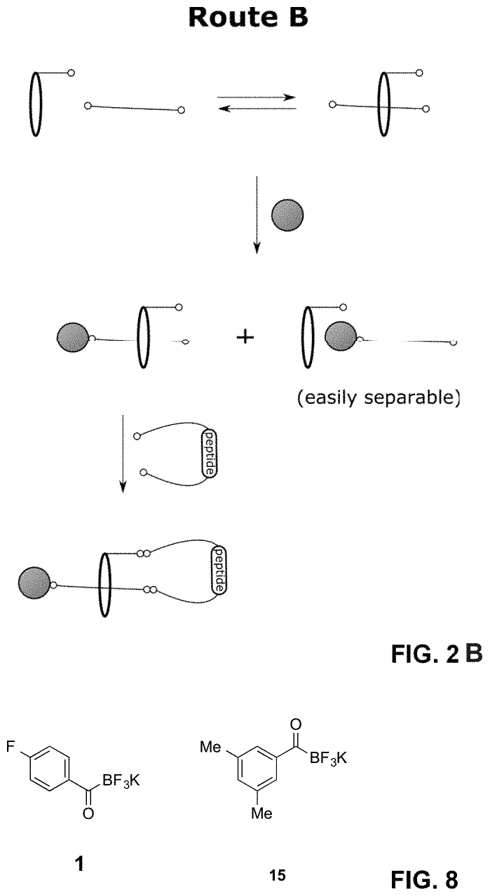

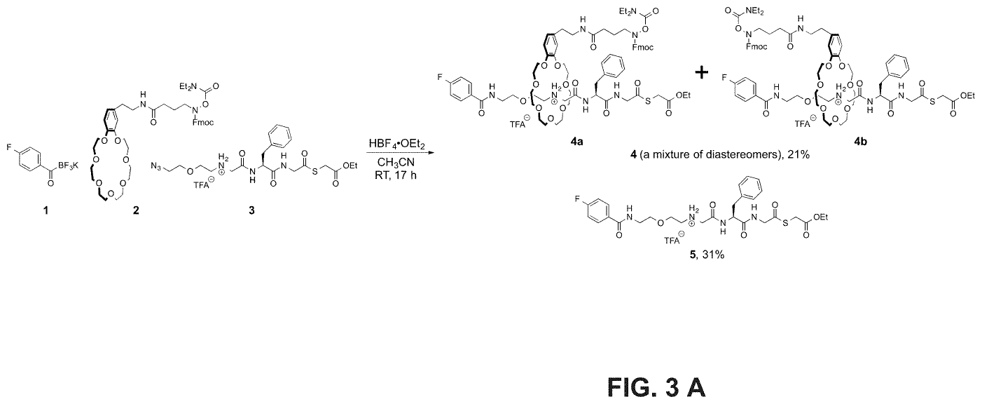

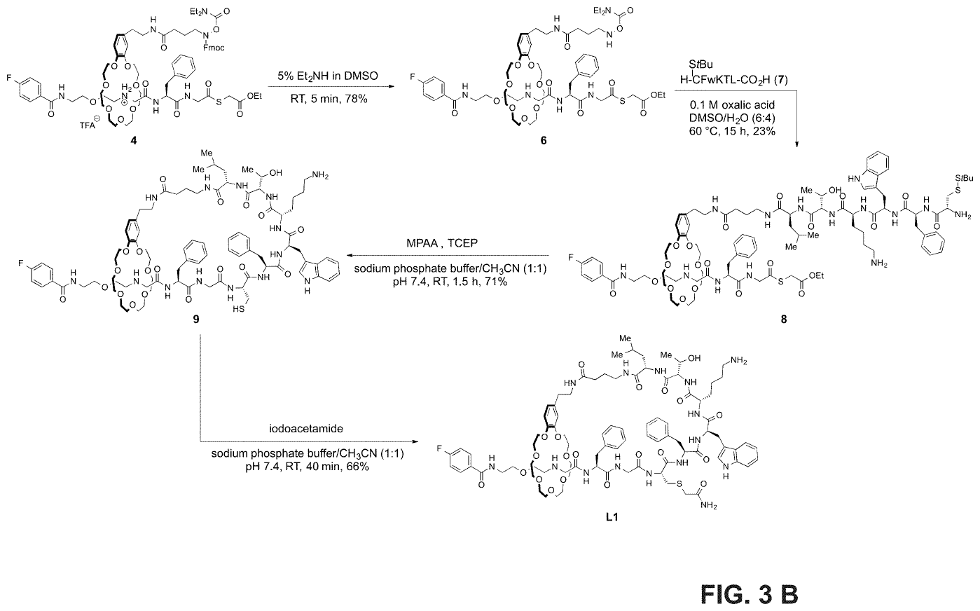

Lasso molecules with incorporated peptides were synthesized. The general synthetic strategy is depicted as Route B in FIG. 2. The synthesis of a first preferred embodiment is shown in FIG. 3. The macrocyclic structure is represented by benzo-21-crown-7 (B21C7,1,4,7,10,13,16,19-Benzoheptaoxacycloheneicosin-21-ethyleneamine) conjugated with Fmoc-protected 4-[[[[diethylamino]carbonyl]oxy]amino]-butanoic acid to give the conjugateable branched macrocycle 2. Reaction of 4-fluorophenyl potassium acyltrifluoroborate 1 and azide 3 in presence of the macrocycle 2 led to the diastereomeric mixture of [2]rotaxanes 4a and 4b in a combined yield of 21%. As a side product, the non-threaded adduct 5 was separated in 31% yield. The Fmoc protecting group of 4 was removed with 5% diethylamine in DMSO giving 6 in 78% yield. Subsequently, the hydroxylamine of 6 was deprotected and KAHA-ligated with the terminal α-ketoacid of the CFwKTL-peptide 7 without intermediate purification. 7 was prepared following a previously published protocol (T. G. Wucherpfennig, V. R. Pattabiraman, F. R. P. Limberg, J. Ruiz-Rodriguez, J. W. Bode, Angew. Chem. Int. Ed. 2014, 53, 12248-12252). The cysteine side-chain protecting group of peptido[2]rotaxane 7 was removed and cyclization by native chemical ligation (NCL) was carried out in one step, affording peptide containing lasso compound 9 in 71% yield. Finally, the sulfhydryl group of 9 was alkylated to prevent oxidation or dimerization. The final lasso compound L1 was thus obtained in 66% yield. The structure of L1 was confirmed by various measurements, including comparative measurements with the separately obtained non-threaded branched-cyclic isomer of L1, namely B1 (see FIG. 4). The experiments included 1H-15N HSQC and ROESY NMR measurements, high-resolution mass spectrometry (HRMS), high performance liquid chromatography (HPLC), and analysis of partial proteolytic degradation studies (see below).

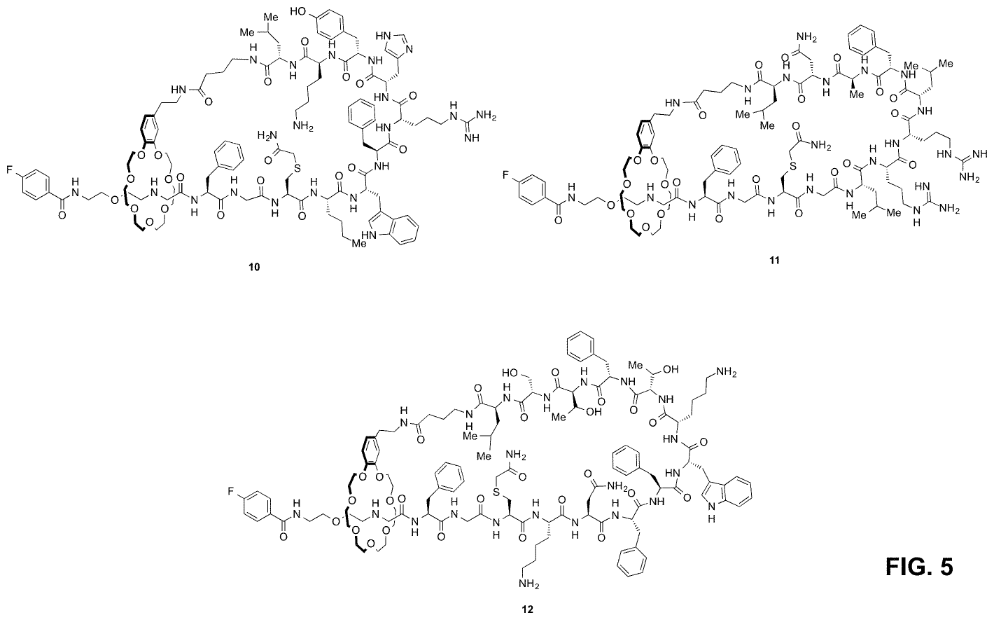

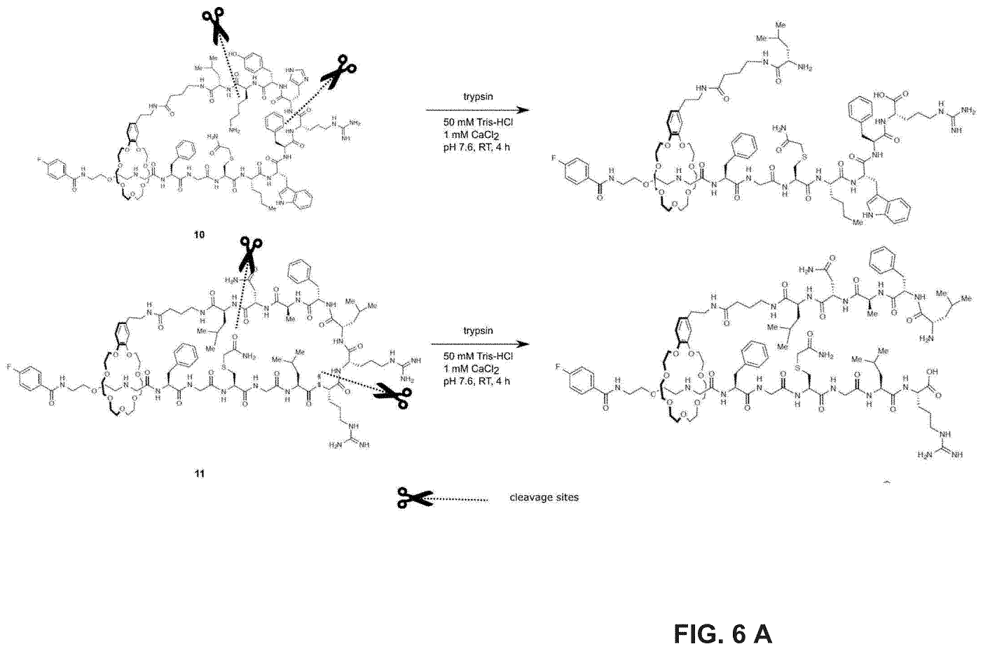

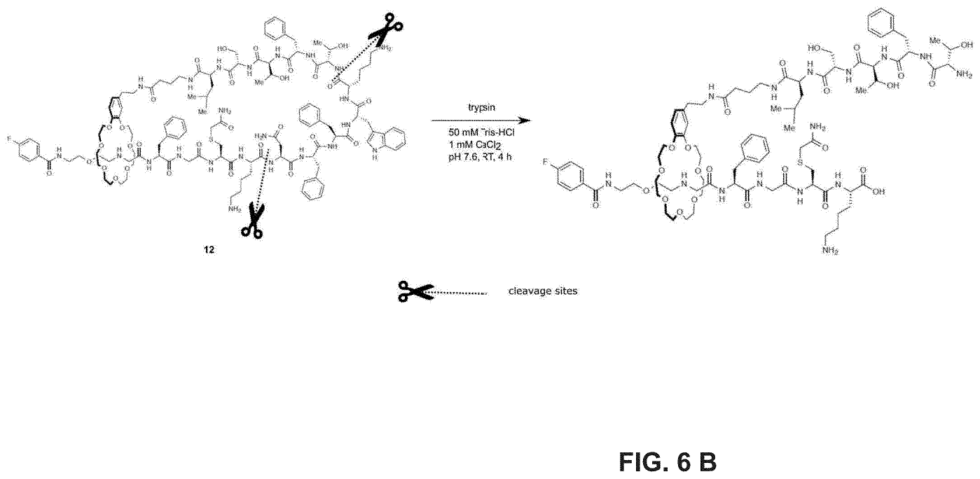

Further, alternative peptide sequences following the same strategy as outlined above were incorporated, namely the sequences Cys-Nle-Trp-Phe-Arg-His-Tyr-Lys-Leu (Seq-ID1), Cys-Gly-Leu-Arg-Arg-Leu-Phe-Ala-Asn-Leu (Seq-ID2), Cys-Lys-Asn-Phe-Phe-Trp-Lys-Thr-Phe-Thr-Ser-Leu (Seq-ID3) (see FIG. 5). The sequences are of medicinal significance, as they were taken from a binder for the Fc region of antibodies, lassomycin, and somatostatin, respectively. However, also nonsensical sequences can be incorporated in an analogous fashion. The lasso structure of all three embodiments was proven by trypsin digestion, which afforded corresponding peptide[2]rotaxanes (see FIG. 6). If the molecules would not have been threaded lassos initially, trypsin digestion would have led to loss of the linear threaded moieties, leading to clearly different mass spectrometric results.

### Example 2

Instead of the macrocycles 1,4,7,10,13,16,19-Benzoheptaoxacycloheneicosin-21-ethyleneamine (see Example 1 above), alternatively 1,4,7,10,13,16,19-Heptaoxacyclodocosan-21-amine, or 1,4,7,10,13,16,19-Heptaoxa-22-azacyclotetracosane were incorporated. To this end, 13 and 14 were synthesized and employed in the synthesis, respectively (see FIG. 7).

Macrocycles 13 and 14 have a larger diameter, compared to 2. As stated above, the size of the stopper moiety has to be large enough to prevent de-threading in significant amounts. Thus, stopper 15 was employed, fulfilling this prerequisite (see FIG. 8). This shows the versatility of the stopper moiety.

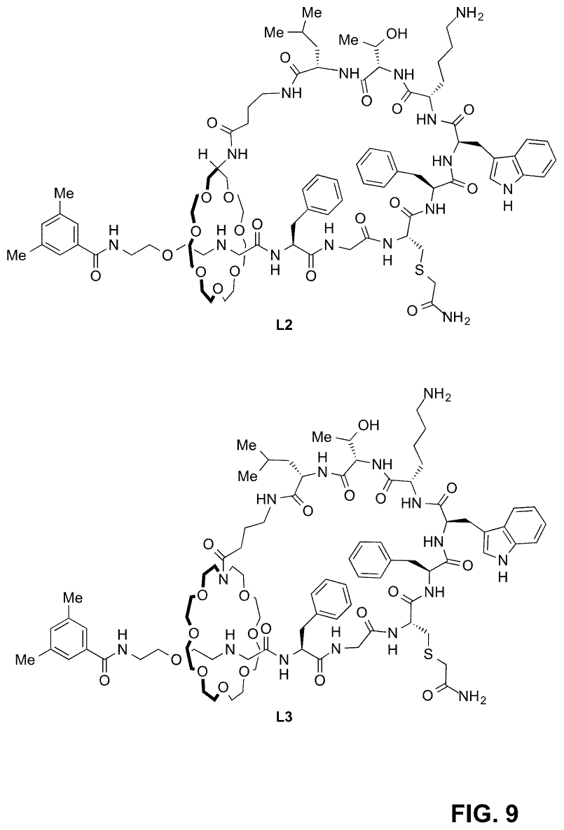

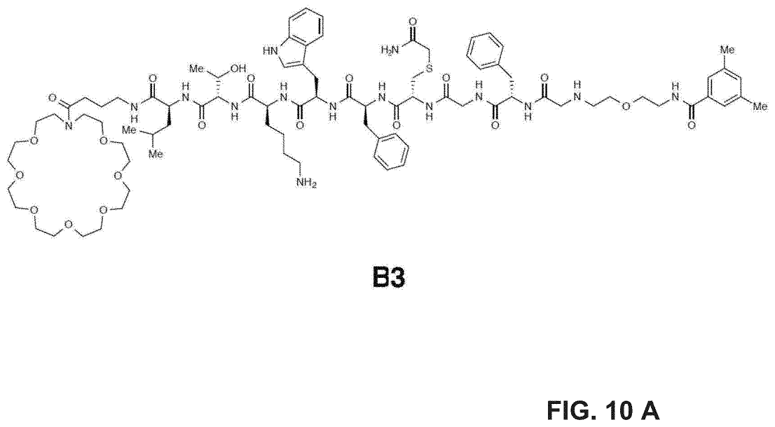

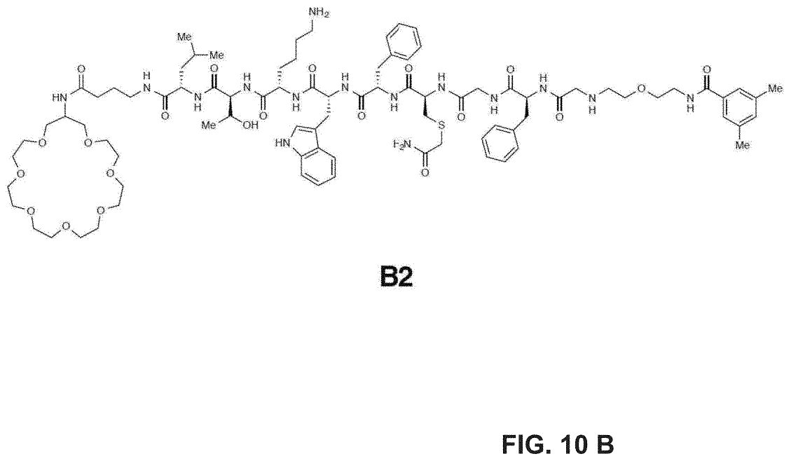

Synthesized structures including the macrocycle 13 or 14, respectively, and the stopper moiety 15 are represented by lasso compounds L2 and L3 (see FIG. 9). The structures of L2 and L3 were confirmed by various measurements, including comparative measurements with the separately obtained non-threaded branched-cyclic isomer of L2 and L3, namely B2 and B3, respectively (see FIG. 10). The analyses further included 1H-15N HSQC and ROESY NMR measurements, high-resolution mass spectrometry (HRMS), high performance liquid chromatography (HPLC), and analysis of partial proteolytic degradation studies.

### Example 3

Some lasso peptides, such as MccJ25, are known to possess a number of favorable properties, among others increased thermal, proteolytic, general stability in biological media such as serum. In proof of concept studies the thermal, proteolytic as well as the stability in serum of L1, L2, and L3 was tested.

Heating to 95° C. for 8 h did not lead to any de-threading of the lasso peptides L1, L2, or L3, as monitored by HPLC.

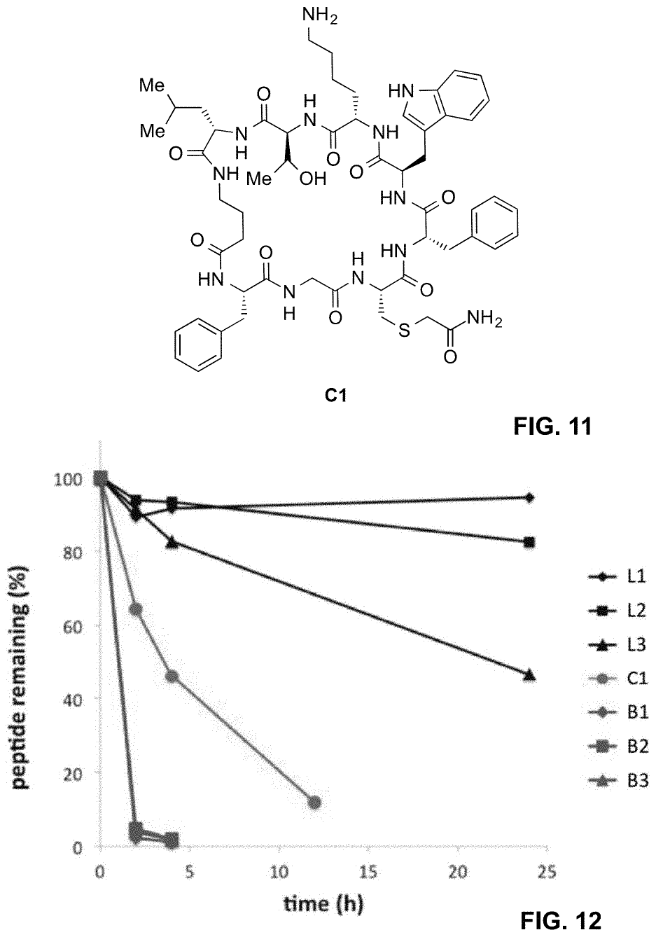

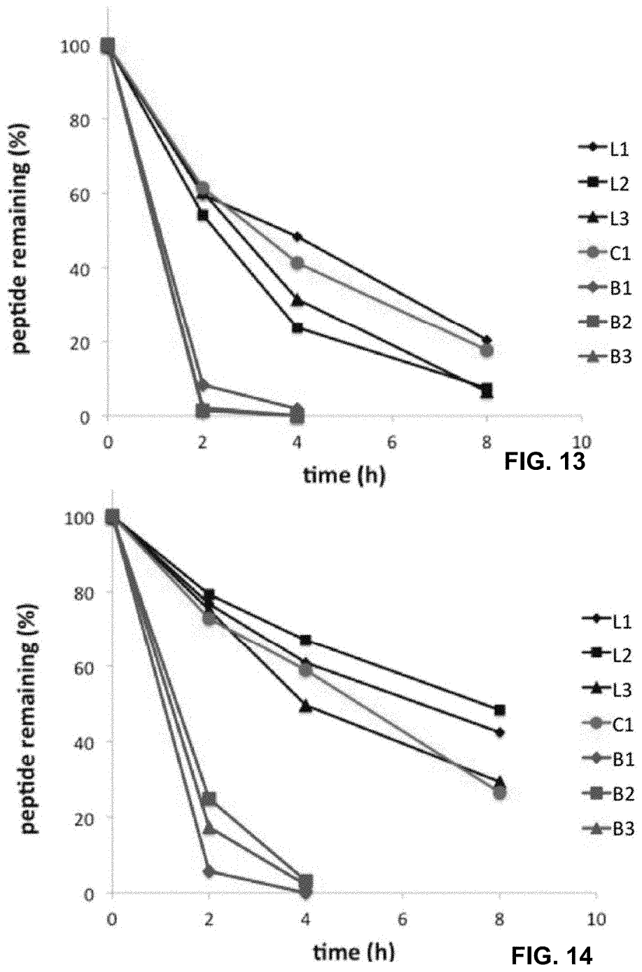

Proteolytic stability of lasso compounds L1, L2, and L3 in comparison to their branched macrocyclic compounds B1, B2, and B3 and the cyclic peptide analogue C1 (see FIG. 11) was tested in separate experiments by incubation for 24 h with chymotrypsin (FIG. 12), trypsin (FIG. 13) and proteinase K (FIG. 14). L1-L3 showed significantly greater stability towards all three proteases compared to B1-B3. In case of trypsin and proteinase K, C1 was similarly stable as L1-L3. In case of chymotrypsin L1-L3 were more stable than C1.

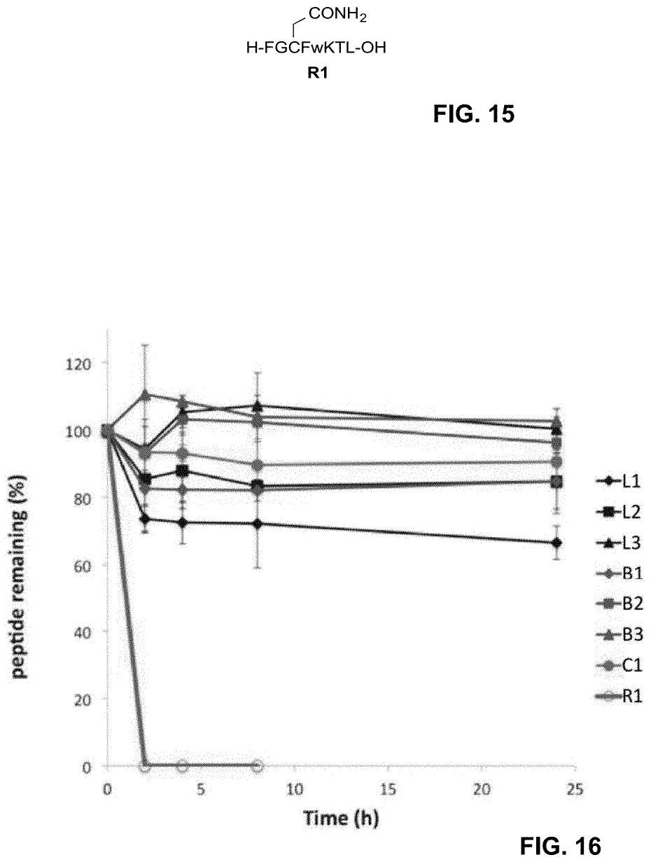

Serum stability of compounds L1, L2, and L3 in comparison to their branched macrocyclic compounds B1, B2, and B3, the cyclic peptide C1, and the linear C- and N-terminally unmasked derivative R1 (see FIG. 15) was tested by incubation for 24 h with human serum (FIG. 16). While R1 was degraded rapidly and completely, all other compounds showed good stability with more than 80% remaining compound at the final time point, except for L1, which was degraded to a final concentration of approximately 70%.

LIST OF REFERENCE SIGNS AND ABBREVIATIONS

| | | | |
|---|---|---|---|
| MccJ25 | Microcin J25 | MPAA | 4-mercaptophenylacetic |
| RGD | arginine-glycine-aspartate | | acid |
| NMR | nuclear magnetic resonance | TCEP | Tris(2- |
| | spectroscopy | | carboxyethyl)phosphine. |
| Fmoc | 9-fluorenylmethoxycarbonyl | HRMS | high resolution mass |
| DMSO | dimethyl sulfoxide | | spectrometry |
| RT | room temperature | Nle | |

1. A method for the synthesis of a molecular lasso structure consisting of a cyclic moiety and of a linear moiety, wherein the linear moiety is covalently attached to the cyclic moiety and with its free end is partially threaded through the orifice formed by the cyclic moiety, including the following steps:
1) provision of a cyclic moiety and of a first linear structural element and establishing conditions in which at least a fraction of the first linear structural element is threaded through the orifice of the cyclic moiety;
2) covalently attaching a stopper element preventing de-threading of the first linear structural element to one terminal end of the first linear structural element;
3) separating unthreaded moieties from threaded moieties, by at least one of chemical or physical separation and using essentially only the threaded molecular assemblies for further reaction;
4) reacting the threaded molecular assemblies with a bifunctional second linear structural element so that the second linear structural element is covalently attached to the first linear structural element at or close to its end opposite to the end where the stopper is attached, and so that the second linear structural element is directly or indirectly covalently attached to the cyclic moiety
with the proviso that a bifunctional second linear structural element can also first be reacted with a cyclic moiety so that the second linear structural element is directly or indirectly covalently attached to the cyclic moiety, prior to, during, or after any of steps 1) or 2), and that in step 4) the threaded molecular assemblies are reacted with the bifunctional second linear structural element directly or indirectly covalently attached to the cyclic moiety so that the second linear structural element is further covalently attached to the first linear structural element at or close to its end opposite to the end where the stopper is attached.

2. The method according to claim 1, wherein
step 3) involves separating unthreaded cyclic moieties and first linear structural elements from threaded molecular assemblies, in which threaded molecular assemblies the first linear structural element with said stopper element is threaded through the cyclic moiety, by at least one of chemical or physical separation and using essentially only the threaded molecular assemblies for further reaction,
or wherein in step 4) the bifunctional second linear structural element is attached with one free end to the corresponding free end of the first linear structural element, and the bifunctional second linear structural element is attached with the other free end to a functional group provided on the cyclic moiety.

3. The method according to claim 1, wherein the cyclic moiety is a ring comprising polyether building blocks.

4. The method according to claim 3, wherein the cyclic moiety is based exclusively on building blocks selected from the group consisting of at least one of: 1,3 or 1,2 propylene-diol, 1,2 ethylene-diol, methylene-diol, benzene-1,2-diol, or is based on these building blocks as well as further based on one or only one building block selected from the group consisting of: methanolamine, ethanolamine, propanolamine, ethylenediamine, 1,3-diaminopropane, 2-aminophenol, benzene-1,2-diamine.

5. The method according to claim 4, wherein the cyclic moiety is selected from the group consisting of: 1,4,7,10,13,16,19-Benzoheptaoxacycloheneicosin-21-ethylene amine, 1,4,7,10,13,16,19-Heptaoxacyclodocosan-21-amine, 1,4,7,10,13,16,19-Heptaoxa-22-azacyclotetracosane, (1,4,7,10,13,16,19-heptaoxacyclohenicosan-2-yl)methanamine 2 (2,3,5,6,8,9,11,12,14,15,17,18,20,21-tetradecahydrobenzo[b][1,4,7,10,13,16,19,22]octaoxacyclotetracosin-24-yl)ethan-1-amine, and mixtures thereof.

6. The method according to claim 1, wherein the cyclic moiety is a crown ether.

7. The method according to claim 1, wherein the cyclic moiety has a diameter of its orifice in the range of 25-45 Å.

8. The method according to claim 1, wherein the first linear structural element in a portion to be located opposite to the stopper element comprises a bulky structural element.

9. The method according to claim 1, wherein the first linear structural element comprises at least one heteroatom in a central region thereof allowing for the establishment of conditions in step 1) under which a non-covalent bond is established in the orifice between the cyclic moiety and said heteroatom.

10. The method according to claim 1, wherein the stopper element is selected to be a structural moiety having an aromatic group.

11. The method according to claim 1, wherein the first linear structural element comprises at least one amino acid building block or at least one building block selected from the group consisting of: methanolamine, ethanolamine, propanolamine, ethylenediamine, 1,3-diaminopropane, 2-aminophenol, benzene-1,2-diamine.

12. The method according to claim 1, wherein the first structural element comprises an activated end for reaction with the stopper element, and a second non-activated end.

13. The method according to claim 1, wherein the second linear structural element is a linear peptide.

14. A molecular lasso structure consisting of a cyclic moiety and of a linear moiety, wherein the linear moiety is covalently attached to the cyclic moiety and is partially threaded through the orifice formed by the cyclic moiety, obtained using a method according to claim 1.

15. The molecular lasso structure according to claim 11 as a medicament.

16. The method according to claim 1, wherein
in step 4) the bifunctional second linear structural element is attached with one free end to the corresponding free end of the first linear structural element, and the bifunctional second linear structural element is attached with the other free end to a functional group provided on the cyclic moiety, wherein the functional group provided on the cyclic moiety is provided as a side chain.

17. The method according to claim 16, wherein the side chain is an amino-group carrying side chain.

18. The method according to claim 1, wherein the cyclic moiety is a ring comprising polyether building blocks, the building blocks being selected from the group consisting of at least 1 of: 1,3 or 1,2 propylene-diol, 1,2 ethylene-diol, methylene-diol, benzene-1,2-diol.

19. The method according to claim 18, wherein the building blocks are substituted by amino, methylamino, ethylamino groups so as to allow for covalent attachment of the second linear structural element.

20. The method according to claim 1, wherein the cyclic moiety is a catechol crown ether.

21. The method according to claim 1, wherein the cyclic moiety has a diameter of its orifice in the range of 30-40 Å.

22. The method according to claim 1, wherein the first linear structural element in a portion to be located opposite to the stopper element comprises a bulky structural element in the form of a side chain.

23. The method according to claim 22, wherein the side chain includes an aromatic group.

24. The method according to claim 1, wherein the first linear structural element in a portion to be located opposite to the stopper element comprises a bulky structural element, wherein the bulky structural element is selected from the group consisting of: phenylalanine, tryptophan, tyrosine, β-amino acids containing bulky side chains, bulky branched-alkyl chains
or wherein the bulky sidechains and/or the bulky branched-alkyl chains are preferably selected from the group consisting of leucine, valine, and isoleucine.

25. The method according to claim 1, wherein the first linear structural element comprises at least one heteroatom in a central region thereof allowing for the establishment of conditions in step 1) under which a non-covalent bond is established in the orifice between the cyclic moiety and said heteroatom, wherein the non-covalent bond is selected from the group consisting of hydrogen bond, ionic bond, or a combination thereof and wherein the heteroatom under the said conditions carries a partial or full positive charge for interaction with heteroatoms in the cyclic moiety, said heteroatoms in the cyclic moiety being selected from the group consisting of oxygen atoms, sulfur atoms, nitrogen atoms.

26. The method according to claim 1, wherein the stopper element is selected to be a structural moiety having an aromatic group, based on benzoic acid, unsubstituted or substituted with one, two, or more aliphatic, including methyl or ethyl groups, and/or halogen atoms, and/or ether groups, including polyether groups including as PEG-substituents on phenols, ether, ester, nitrile, and nitro groups.

27. The method according to claim 1, wherein the stopper element is selected from the group consisting of: mono fluoro benzoic acid, benzoic acid, methylbenzoic acid, dimethylbenzoic acid, bromo benzoic acid, iodo benzoic acid, and 2-phenylacetic acid, which are unsubstituted or substituted, in the latter case preferably with groups including ether, ester, nitrile, and nitro groups,
or wherein for reaction in step 2) the stopper element is provided as an activated compound, including systems with alkyne, maleimide, alkene, azide, and aldehyde/ketone structural elements, or as a acyltrifluoro or borate compound, including an acyltrifluoroborate.

28. The method according to claim 1, wherein the first structural element comprises an activated end for reaction with the stopper element, and a second non-activated end, wherein the activated end takes the form of a group selected from the following group nitrile, azido, thiol, alkene, in particular C1-C6 alkene, alkyne in particular C1-C6 alkyne, hydroxylamine.

29. The method according to claim 1, wherein the second linear structural element is a linear peptide, having 3-15 amino acids, or having 4-10 or 5-8 amino acids.

30. The method according to claim 1, wherein the second linear structural element is a linear peptide with known therapeutic effect.
